(19) United States
(12) Patent Application Publication
(10) Pub. No.: US 2011/0236902 A1
(43) Pub. Date: Sep. 29, 2011

(54) TESTING A PATIENT POPULATION HAVING A CARDIOVASCULAR CONDITION FOR DRUG EFFICACY

(75) Inventors: Angela Soito, Oakland, CA (US); John B. Simpson, Woodside, CA (US)

(73) Assignee: TYCO HEALTHCARE GROUP LP, MANSFIELD, MA (US)

(21) Appl. No.: 13/154,057

(22) Filed: Jun. 6, 2011

Related U.S. Application Data
(63) Continuation of application No. 11/367,719, filed on Mar. 6, 2006.
Application No. 11/367,719 is a continuation-in-part of application No. 11/199,370, filed on Aug. 9, 2005.
Application No. 11/199,370 is a continuation-in-part of application No. 11/010,833, filed on Dec. 13, 2004.

(51) Int. Cl. C12Q 1/68; C12Q 1/24

(52) U.S. Cl. 435/6.13; 435/30

(57) ABSTRACT

Lumenectomy material is tested to determine the efficacy of a test drug in a patient population having a cardiovascular condition. The material is removed from at least a first and a second patient and tested for one or more markers of a cardiovascular condition. The first patient is administered the test drug, and the second patient is administered a placebo. At a later date, more lumenectomy material is removed and tested for the same marker or markers. The presence, absence or amount of the markers is compared in the first patient receiving the drug and the second patient receiving the placebo to determine whether the drug is effective in the patient population. The drugs tested include drugs believed to be effective in treating a cardiovascular condition. The markers used can include any marker that can indicate the effectiveness of the drug being tested.

### CROSS-REFERENCES TO RELATED APPLICATIONS

This application is a continuation of U.S. patent application Ser. No. 11/367,719, filed Mar. 6, 2006, which is a continuation-in-part of U.S. patent application Ser. No. 11/199,370, filed Aug. 9, 2005, which is a continuation-in-part of U.S. patent application Ser. No. 11/010,833, filed Dec. 13, 2004, the contents of each of which are hereby incorporated by reference herein.

### TECHNICAL FIELD OF THE INVENTION

This invention is related to the area of disease diagnosis and prognosis. In particular, it relates to testing for markers in lumenectomy samples of a patient population administered a test drug, the results of the testing being determinative of the effectiveness of the drug.

### BACKGROUND OF THE INVENTION

Cardiovascular disease frequently arises from the accumulation of atheromatous material on the inner walls of vascular lumens, particularly arterial lumens of the coronary and other vasculature, resulting in a condition known as atherosclerosis. Atherosclerosis occurs naturally as a result of aging, but may also be aggravated by factors such as diet, hypertension, heredity, vascular injury, and the like. Atheromatous and other vascular deposits restrict blood flow and can cause ischemia which, in acute cases, can result in myocardial infarction. Atheromatous deposits can have widely varying properties, with some deposits being relatively soft and others being fibrous and/or calcified. In the latter case, the deposits are frequently referred to as plaque.

One conventional treatment for cardiovascular disease is the use of stents. Endolumenal stents are commonly used to treat obstructed or weakened body lumens, such as blood vessels and other vascular lumens. Once deployed in the blood vessel, the stent can remain in the body lumen where it will maintain the patency of the lumen and/or support the walls of the lumen which surround it. One factor impeding the success of stent technology in endolumenal treatments is the frequent occurrence of in-stent restenosis, characterized by proliferation and migration of smooth muscle cells within and/or adjacent to the implanted stent, causing reclosure or blockage of the body lumen.

Atherosclerosis and restenosis can be treated in a variety of ways, including drugs, bypass surgery, and a variety of catheter-based approaches which rely on intravascular debulking or removal of the atheromatous or other material occluding a blood vessel. Of particular interest to the present invention, a variety of methods for cutting or dislodging material and removing such material from the blood vessel have been proposed, generally being referred to as atherectomy procedures. Atherectomy catheters intended to excise material from the blood vessel lumen generally employ a rotatable and/or axially translatable cutting blade which can be advanced into or past the occlusive material in order to cut and separate such material from the blood vessel lumen. In particular, side-cutting atherectomy catheters generally employ a housing having an aperture on one side, a blade which is rotated or translated by the aperture, and a balloon to urge the aperture against the material to be removed.

Although atherectomy catheters have proven very successful in treating many types of atherosclerosis and in-stent restenosis, improved atherectomy catheters and methods are continuously being pursued. For example, many currently available side-cutting atherectomy catheters have difficulty in capturing occluding material in the cutting aperture. To facilitate material capture, the cutting aperture is frequently elongated to increase the area into which the material can penetrate. Such elongation typically requires an equivalent lengthening of the cutter housing. Since most cutter housings are rigid, such lengthening makes it more difficult to introduce the distal end of the catheter through tortuous regions of the vasculature.

Another shortcoming of many currently available atherectomy catheters is that they typically require a balloon positioned opposite the cutting window to urge the cutting window into contact with occluding material. Such balloons, however, unduly increase the size of the distal portion of the catheter. Even with the balloon, the amount of material that can be removed by conventional atherectomy catheters is limited by the size of the cutting window. Other disadvantages of some catheters include cutting elements with less than ideal hardness, inadequate storage space within the catheter for containing removed material, sub-optimal guide wire lumens, and/or the like. In addition, the available atherectomy catheters generally provide material insufficient in quantity and/or quality for testing by many histological, array, proteomic or other biochemical or molecular methods. For example, in one report a device and method available to the artisan collected less than about 50 mg of lumenectomy material. (Safian et al., Circulation 82: 305-307 (1990)). This amount of material is not typically enough to carry out more than one test, or is insufficient to successfully carry out a number of diagnostic tests available to the physician or researcher.

Recently atherectomy catheters have been developed which can access small, tortuous regions of the vasculature and remove atheromatous and other occluding materials from within blood vessels and stents in a controlled fashion. In particular, these atherectomy catheters facilitate capturing and invagination of atheromatous materials. Particularly, these catheters are capable of in vivo capturing and removing of continuous lumenectomy material strands of sufficient quantity and quality for testing in vitro. These catheters and methods for their use are adaptable for use in a variety of body lumens, including but not limited to coronary and other arteries.

There is a continuing need in the art to develop new methods of testing new drugs, particularly ways to test so that mortality is not the endpoint at which whether or not the drug has been effective is determined. Waiting for a mortality endpoint significantly delays the time a drug can get to market. It would be advantageous to employ the information that can be yielded from an analysis of lumenectomy material removed from a patient having a cardiovascular condition in order to determine whether the drug is effective in treating the condition.

The present invention provides some methods for testing drugs using vascular tissue and lumenectomy material removed from patients having a cardiovascular condition.

### SUMMARY OF THE INVENTION

One aspect of the invention provides a method of screening for drug efficacy in a population of patients having cardiovascular disease comprising removing a first sample of lumenectomy material from a first location in vascular lumens of a first and a second patient population, testing the first sample in a first test for the presence or absence or amount of a marker, administering a drug to the first patient population and a placebo to the second patient population, removing a second sample of lumenectomy material from a second location in vascular lumens of the first and second patient population, testing the second sample in a second test for the presence or absence or amount of the marker, and evaluating the efficacy of the drug on the basis of comparing the presence or absence or amount of the marker in the first and second tests in the first patient population administered the drug versus the second patient population administered the placebo.

Another aspect of the invention provides a method of screening for drug efficacy in a patient having cardiovascular disease comprising removing a first sample of lumenectomy material from a first location in a vascular lumen of the patient, testing the first sample in a first test for the presence or absence or amount of a marker, administering a drug to the patient, removing a second sample of lumenectomy material from a second location in a vascular lumen of the patient, testing the second sample in a second test for the presence or absence or amount of the marker, and evaluating the efficacy of the drug on the basis of comparing the presence or absence or amount of the marker in the first and second tests.

Yet another aspect f the invention is a method of screening for drug efficacy in a population of patients having cardiovascular disease comprising removing a first sample of lumenectomy material from a first location in a vascular lumen of at least a first patient and a second patient, testing the first samples in a first test for the presence or absence or amount of a marker, administering a drug to the first patient and a placebo to the second patient, removing a second sample of lumenectomy material from a second location in a vascular lumen of at least the first patient and the second patient, testing the second samples in a second test for the presence or absence or amount of the marker, and evaluating the efficacy of the drug on the basis of comparing the presence or absence or amount of the marker in the first and second tests for the first patient administered the drug compared to the second patient administered the placebo.

This and other embodiments which will be apparent to those of skill in the art upon reading the specification provide the art with methods for detection, diagnosis, and prognosis of diseases.

### DETAILED DESCRIPTION OF THE INVENTION

The inventors have developed methods for testing the effectiveness of new drugs to treat cardiovascular conditions. Lumenectomy material is excised from the vascular lumens of patients in a first sample and analyzed for one or more markers. The marker or markers selected indicate the status of a cardiovascular condition in the patient. One or more patients in a first group are given a test drug, and in parallel as a control, one or more patients in a second group are given a placebo. At some time point later, a second sample of lumenectomy material is removed from the patients and analyzed for the presence, absence or amount of the one or more markers. The results of the “drug” patients and the “placebo” patients are compared to determine whether the test drug is effective in treating the cardiovascular condition.

Additionally, a single patient can be used for a study to determine the effectiveness of a drug by removing a first sample of lumenectomy material from a first vascular lumen in the patient and testing that sample for a marker, administering a drug to the patient, and removing a second sample of lumenectomy material from the patient from a second vascular lumen in the patient, in other words from a different location in the patient than the first lumenectomy sample was retrieved, testing the second sample for the same marker as the first and comparing the marker results in the two samples.

The patient population can be a few patients, a dozen patients, or hundreds of patients. The population can be defined as patients having or susceptible to a cardiovascular condition. The lumenectomy samples are withdrawn from each patient in a similar fashion. The locations from which the lumenectomy samples are withdrawn may depend on such parameters as where the patient has diseased vasculature, or from a pre-determined location in all patients such as a particular appendage.

The cardiovascular condition can be any cardiovascular condition. The main cardiovascular conditions of interest include atherosclerosis or restenosis, conditions that have many symptoms and repercussions to the overall health of the patient, but which manifest their presence in the patient by being localized in the vasculature and as such exerting their primary effects on blood flow and heart function from that location. Removed lumenectomy material from the atherosclerotic or restenotic regions of the patient's vasculature can be tested for the presence, absence or amount of one or more markers of atherosclerosis or restenosis, or vascular endothelial cell proliferation generally. In general the lumenectomy material can be atherosclerotic tissue, restenotic tissue, or any vascular tissue retrievable from a vascular lumen.

In general the drug should be able to reverse, prevent, or stabilize a cardiovascular condition. Some exemplary test drugs can include any drug which a research institution or individual has reason to believe may be effective in reducing, ameliorating, or reversing a cardiovascular condition. Examples of such drugs include the following: monoclonal antibody EP-SC7, fibroblast growth factor-saporin mitotoxin, magnolol, telebermin, probucol, and many other drugs that have been thought at one time or another to possess potential for effectiveness in treating cardiovascular conditions, or which shows new promise in treating cardiovascular conditions.

The lumenectomy material is removed from the patient by any means possible to remove the material from a vascular lumen and still preserve the integrity of the patient's vasculature. Generally greater than 50 mg of lumenectomy material is removed from each patient for testing. Accordingly, the lumenectomy material, or vascular tissue, can be excised using a percutaneous surgical procedure in which a catheter is placed in the vessel, and a cutter is engaged to cut away lumenectomy material from the lumen wall, and direct is to a collection chamber or otherwise deliver the material to the outside of the patient for preservation, storage, or testing. Exemplary catheters equipped for this task include the Silver Hawk™ excision devices, and those devices described generally in U.S. Ser. No. 11/010,833 and U.S. Ser. No. 11/199,370. Lumenectomy catheters which can be used to collect the samples of the present invention are described in U.S. application publication no. 20050177068, the disclosure of which is expressly incorporated herein. Other lumenectomy catheters which provide sufficient material for testing may also be used. In certain embodiments the amount of material collected can be about 1 mg to about 2000 mg, more typically the amount of material can be about 1 mg to about 100 mg, about 100 mg to about 200 mg, about 200 mg to about 300 mg, about 300 mg to about 400 mg, about 400 mg to about 500 mg, about 500 mg to about 600 mg, about 600 mg to about 700 mg, about 700 mg to about 800 mg, or about 800 mg up to about 2000 mg. The material excised from the body lumen will vary in length and will depend on the catheter configuration, the type of material removed, the body lumen, and the like. However, in certain embodiments, the material will be in the form of continuous strands that have a substantially consistent depth and width of lumenectomy material cuts. The material is typically longer than the length of the cutting window (but it may be shorter), and typically has a length of at least about 2.0 mm, although the length may be between about 0.5 cm up to about 10 cm or longer in length. Advantageously, the planing action of the catheter provides a material lumenectomy material structure that reflects the actual in vivo lumenectomy material structure, and provides information about larger portions of the disease state of the body lumen.

In a population study two groups of patients can be identified. The first group of patients can be the “drug” group who receive the test drug. The second group of patients can be the “placebo” group who receive the placebo. From the first group at least one patient is selected, and from the second group at least one patient is also selected. From the first group (or first patient) a first lumenectomy sample is removed. The sample is removed so that it is preserved for testing, or placed into an environment for testing. From the second group (or second patient) a second lumenectomy sample is removed and preserved or tested also. The types of tests employed for a particular procedure depend largely on the marker or markers being looked at. Thus the nature of the marker will dictate in large part what types of tests are performed on the lumenectomy material.

Markers which can be tested are any for which an association has been established between the marker and the disease or imminent onset of the disease. Markers can be, for example, proteins, enzymes, or RNAs. The marker can be the presence or absence or amount of a substance or an increased or decreased level of the substance. The material collected from the body lumen is typically a continuous strip of lumenectomy material that may be longer than the cutting window of the lumenectomy catheter. This material can provide a sufficient amount of sample material of a quality and quantity that can be used for one or more of genomic screening, DNA hybridization, RNA hybridization, gene expression analysis, PCR amplification, proteomic testing, drug efficacy screening, protein marker detection, DNA marker detection, RNA marker detection, histological testing, histopathology, cytopathology, cell and lumenectomy material type analysis, biopsy, or the like. In addition, the material collected may be sufficient in amount and quality for testing for one or more of the presence of a DNA, an RNA, or a protein marker.

Generally the markers may be in the category of apoptotic markers, cell cycle proteins, transcriptional factors, proliferative markers, endothelial growth factors, adhesion molecules, cytokines, chemokines, chemokine receptors, inflammation markers, coagulation factors, fibrinolytic factors, oxidative stress related molecules, extracellular matrix molecules, interleukins, growth factors, glycoproteins, proteoglycans, cell-surface markers, serum markers, or immune factors. Other types of markers which are established as associated with the diseases may be used as well.

Specific markers which may be used include C-reactive protein, interleukin-6, and/or intracellular adhesion molecule-1 for depression; angiotensin II, aldosterone, and/or atrial natriuretic factor for hypertension; lumenectomy material factor pathway inhibitor, plasminogen activator inhibitor-1, triglycerides, and/or apolipoprotein B for hyperlipidemia; triglycerides for insulin resistance; low density lipoprotein, Remnant-like particles-cholesterol and/or triglycerides for diabetes; triglyceride-rich lipoproteins for kidney damage. Other markers as are known in the art and which are associated with specific diseases can be used as well, without limitation.

The markers that can be tested for include any marker comprising an amino acid, or any marker comprising a nucleic acid. Thus, the marker can be a peptide, polypeptide, or protein, or a DNA or RNA molecule. The marker can also comprise a cell-surface molecule, particularly a cell surface molecule on a vascular endothelial cell, or other cell located within the cardiovascular system of the patient. The marker can be any marker found in vascular lumenectomy material of humans. Some exemplary markers include the following which can be peptide, polypeptide, protein, nucleic acid (DNA, RNA) markers. In general, any marker that would indicate some information about a cardiovascular condition can be used either alone or in conjunction with other markers in order to determine whether a test drug (administered to the patient) is effective or not. The list of markers in Table 1 below is not intended to be exhaustive of the markers that can be used in the practice of the invention, but rather exemplary.

TABLE 1

MARKERS
a disintegrin-like and metalloprotease (reprolysin type)
actin related protein 2/3 complex, subunit 2
adhesion molecules
albumin
alpha-tocopherol
angiotensin-converting enzyme
Apoptotic markers
ATPase, Na+/K+ transporting, beta 3 polypeptide
ATP-binding cassette, sub-family A (ABC1), member 1
Bak
basic fibroblast growth factor (bFGF)
basic helix-loop-helix domain containing, class B, 2
Bax
BCl-2
Bcl-x
beta thromboglobulin,
B-factor, properdin
bFGF
big endothelin
Biglycan
C reactive protein (CRP),
calmodulin 2 (phosphorylase kinase, delta)
cardiac troponin T (cTnT)
cardiac troponin I (cTnI) alkaline phosphatase
cathepsin B
CCR2
CCR3
CD11a
CD11b
CD18
CD19 antigen
CD20
CD3
CD31
CD36
CD36 antigen (thrombospondin receptor)
CD4
CD40
CD62P
CD68
CD8
Cell-surface markers
chemokine C-X-C motif, granulocyte chemotactic protein 2
Chemokine fractaline
Chemokine receptors
Chemokines
chemotactic proteins
chitinase 3-like 2
chondroitin/dermatan sulfate proteoglycan (PG40) core
clade E
Coagulation factors
Collagen
collagen, type I, alpha 1
collagen, type I, alpha 2
collectin sub-family member 12
colony stimulating factor 1 receptor
Complement C3
complement component 1, r subcomponent
Complement proteins
creatine isoenzyme MB
creatine kinase (CK)
CREB
CX3C
CX3CR1
Cyclin A
Cyclin B
Cyclin D
Cyclin E
cyclin-dependent kinase inhibitor lA (p21, Cip1)
cyclin-dependent kinase inhibitor 2A
cysteine-rich, angiogenic inducer, 61
cytochrome P450, subfamily I (dioxin-inducible)
Cytokines
decorin
dehydro-thromboxane B2, thromboxane A2,
Deorin
DVS27-related protein
E selectin
E2F
early growth response 1
EDG r
Elastin
endomucin-2
endothelial cell markers
endothelial cells
endothelial cell-specific molecule 1
Endothelial dysfunction/Injury (s-ICAM, P-selectin)
Endothelial growth factors
endothelin-1
Eotaxin
Ephrins
erythrocyte alpha-tocopherol
E-selectin
Extracellular matrix molecules (such as calcium, collagen)
fatty acid binding protein 4, adipocyte
fatty acid binding protein 5 (psoriasis-associated)
FGF receptor
fibronectin
Fibrinogen
Fibrinolytic factors
fibroblast growth factor 7 (keratinocyte growth factor)
FKBP12
fractalkine
fractalkine, inducible cytokine subfamily D (Cys-X3-Cys)
G proteins
gamma interferon
glutamine-fructose-6-phosphate transaminase 2
glycoprotein (transmembrane) nmb
Glycoprotein receptoron platelets
Glycoproteins
GpIIb-IIIa
growth factor receptor-bound protein 2
Growth factors
guanine nucleotide binding protein, beta polypeptide 1
H factor 1 (complement)
heterogeneous nuclear ribonucleoprotein F
high density lipoprotein, erythrocytes
Human chondroitin sulfate proteoglycan core protein
hyaluronan synthase 2
hyaluronan-mediated motility receptor (RHAMM)
Hyaluronan
ICAM-1
IF-gamma
IL-1
IL-6
IL-8
Inflammatory markers (CRP, IL-6, MIP-1β, TNFα-R, RANTES, MPO)
insulin-like growth factor binding protein 4
integral membrane protein 2A
integral membrane protein 2B
Integrin
integrin, alpha 2b
integrin, alpha 4 (antigen CD49D, alpha 4 subunit of VLA-4 receptor)
integrin, alpha M (complement component receptor 3, alpha)
integrin, beta 3 (platelet glycoprotein IIIa, antigen CD61)
intercellular adhesion molecule 1 (CD54)
interferon, gamma
interferon, gamma-inducible protein 16
interleukin 1 receptor, type I
interleukin 1, alpha
interleukin 18 (interferon-gamma-inducing factor)
interleukin 2 receptor, beta
interleukin 6 (interferon, beta 2)
interleukin 8
interleukin-1 (IL-1)
interleukin-18
interleukin-1-beta
interleukin-6 (IL-6)
interleukins
isoleucine-tRNA synthetase
jun B proto-oncogene
Ki-67
KLF5/BTEB2
Lamin receptor
laminin, alpha 4
LFA-1
L-homocysteine
Lipid (cholesterol, cholesterol ester)
lipopolysaccharide (LPS), troponin T
Lipoxygenase
LPPLA2
lymphocytes function associated antigen-1 (LFA-1)
MAC-1
macrophage migration inhibitory factor
macrophage scavenger receptor 1
macrophages
major histocompatibility complex, class I, C
mannosidase, alpha, class 1A, member 1
MARCKS-like protein
matrix metalloproteinase 1 (interstitial collagenase)
matrix metalloproteinase 10 (stromelysin 2)
matrix metalloproteinase 2
matrix metalloproteinase 3 (stromelysin 1, progelatinase)
matrix metalloproteinase-9
matrix metalloproteinases
MCP-1
Metalloproteinase
metallothionein 2A
microsomal glutathione S-transferase 1
mitochondrial ribosomal protein L51
MMP
MMP-1
MMP-12
MMP-13
MMP-2
MMP-3
MMP-7
MMP-8
MMP-9
monocyte chemotactic protein 1
monocyte chemotactic protein 2
monocyte chemotactic protein 3
Monocyte colony stimulating factor M-CSF
monocyte inflammatory protein alpha
monocytes
MPO
multiple endocrine neoplasia I
Myeloperoxidase
myosin, heavy polypeptide 11, smooth muscle
myristoylated alanine-rich protein kinase C substrate
neural precursor cell expressed, developmental regulated 5
neutrophils
nicotinamide N-methyltransferase
NO/EDRF
N-terminal pro-brain natriurectic peptide (NT-proBNP)
nuclear factor-kappa B (NF kappaB)
osteopontin
Oxidative stress related molecules (atheronal)
oxidized LDL
oxidized low density lipoprotein (lectin-like) receptor 1
P53
pCRP
PD biomarker
PDGF
PDGF receptor
PDZ domain proteins
pentaxin-related gene, rapidly induced by IL-1 beta
phosphogluconate dehydrogenase
phospholipid scramblase 3
Plaque stability markers (MMPs, PAPP-A)
plasminogen activator inhibitor
plasminogen activator, tissue
plasminogen activator, urokinase
plasminogen activator, urokinase receptor
platelet factor 4
platelet/endothelial cell adhesion molecule (CD31 antigen)
pleiomorphic adenoma gene-like 1
pre-B-cell colony-enhancing factor
pregnancy-associated plasma protein A
Proliferating cell nuclear antigen PCNA
Proliferative markers
prostaglandin-endoperoxide synthase 1
protein phosphatase 1, regulatory (inhibitor) subunit 16B
Proteoglycans
Pro-thrombinogen
p-selectin
RANTES
regulator of G-protein signaling 2, 24 kDa
ribosomal protein S26
selectin
serine (or cysteine) proteinase inhibitor
serine (or cysteine) proteinase inhibitor, clade E
serine (or cysteine) proteinase inhibitor, clade F
serine (or cysteine) proteinase inhibitor, clade H
SH3-domain binding protein 5 (BTK-associated)
sICAM-1
Smooth muscle actin
Smooth muscle proliferative inhibitors
Smooth muscle proliferative promotors
soluble intercellular adhesion molecule-1 (sCAM-1)
solute carrier family 20, member 1
stanniocalcin 2
stromal cell-derived factor 1
sulfotransferase, estrogen-preferring
sushi-repeat-containing protein, X chromosome
syndecan binding protein (syntenin)
TGF-alpha
TGF-beta
TGF-beta receptor
Thy-1 cell surface antigen
Tissue factor
tissue inhibitor of metalloproteinase 1
tissue inhibitor of metalloproteinase 2
TNF
TNF-alpha
TNFa-RII
TNF-beta
transcription elongation factor A (SII), 1
Transcription factor
UDP-glucose ceramide glucosyltransferase
vascular endothelial growth factor (VEGF)
VCAAM-1
Versican
von Willebrand factor

Particular types of tests that can be carried out successfully on the excised lumenectomy material removed by the methods of the present invention include, but are not limited to, enzyme histochemistry, immunohistology, immunocytochemistry, immunoassays, immunofluorescent assays, immunoprecipitation assays, ELISA, flow cytometry, fluorescent activated cell sorting, radioimmunochemistry, electrophoresis, two-dimensional gel electrophoresis, Western blotting, protein sequencing, mass spectrometry, proteomic analysis, and protein microarray analysis. Further, Northern blotting, RNase protection assays, in situ hybridization assays, DNA microarray testing, reverse transcription polymerase chain reaction PCR (RT-PCR), Southern blotting, DNA sequencing, PCR amplification, single strand conformational polymorphism assays, single strand polymorphism (SNP) assays, and serial analysis of gene expression (SAGE) assays can be successfully carried out with the lumenectomy material compositions collected by the disclosed methods.

Prior to testing the harvested material, the material can optionally be placed in a preserving agent, a lumenectomy material fixative, or a preparation agent compatible with a particular test to be run. Agents known in the art for preserving, fixing or preparing the material for later use include, for example, saline, heparinized saline, liquid nitrogen, formalin, a membrane lysis agent, an RNA or DNA preparation agent, and the like. The material can be collected in a single access or can be collected in multiple translumenal accesses in the same patient. Further the material is typically at least one substantially consistent, continuous strip of material that maintains the structure of the material as it was removed from the inner surface of the lumen of the patient. Also, sample material can be collected from one, two, or more sites in the same or a different body lumen of a patient.

The lumenectomy catheters can achieve selective plaque excision, i.e., they can specifically target diseased areas. Thus the samples are enriched in disease markers, relative to serum samples, in which disease markers are diluted with other substances from non-diseased lumenectomy materials. Nonetheless, serum or blood testing may be performed in conjunction with the lumenectomy evaluation, and the results used, for example, to confirm each other.

When a population is studied to determine whether a drug is effective in a plurality of individuals, the first group receives the test drug, and the second group receives the placebo (after the initial analysis of the lumenectomy material in both groups). The drug is given sufficient time to have some effect on the patient, and then a second lumenectomy sample from all the patients in each population is withdrawn. The second sample is tested for the same marker or markers as the first sample, and the difference in both the test drug population and the placebo population are compared to determine whether or not the test drug was effective in treating the cardiovascular condition.

The above disclosure generally describes the present invention. All references disclosed herein are expressly incorporated by reference.

What is claimed is:

1. A method of screening for drug efficacy in a population of patients having cardiovascular disease comprising:
removing a first sample of lumenectomy material from a first location in a vascular lumen of a first and a second patient population;
testing the first sample in a first test for the presence or absence or amount of a marker;
administering a drug to the first patient population and a placebo to the second patient population;
removing a second sample of lumenectomy material from a second location in a vascular lumen of the first and second patient population;
testing the second sample in a second test for the presence or absence or amount of the marker; and
evaluating the efficacy of the drug on the basis of comparing the presence or absence or amount of the marker in the first and second tests in the first patient population administered the drug versus the second patient population administered the placebo,
wherein the lumenectomy material comprises an atherosclerotic tissue, a restenotic tissue, or other tissue excised from a vascular lumen.

2. The method of claim 1, wherein the drug is a drug to reverse, prevent, or stabilize a cardiovascular condition.

3. The method of claim 2, wherein the cardiovascular condition comprises atherosclerosis or restenosis.

4. The method of claim 1, wherein the marker comprises an amino acid.

5. The method of claim 1, wherein the marker comprises a nucleic acid.

6. The method of claim 1, wherein the marker comprises a cell surface molecule.

7. The method of claim 1, wherein the marker comprises a marker found in vascular lumenectomy material.

8. The method of claim 1, wherein removing the lumenectomy material comprises percutaneous surgical excision comprising:
providing a catheter having a rotating cutter, a collection chamber, and a cutting window, the collection chamber being distal to the cutting window, the rotating cutter being movable between a stored position and an exposed position, at least part of the rotating cutter becoming exposed through the cutting window when moving to the exposed position;
exposing the cutter by moving the cutter to the exposed position; and
advancing the catheter in a distal direction to move the rotating cutter through occlusive material in the body lumen, the rotating cutter remaining in the exposed position so that the cutter and the window maintain their orientation with respect to one another when advancing the catheter through the occlusive material, the occlusive material cut by the rotating cutter being directed through the cutting window and distally into the collection chamber as the catheter is advanced in the distal direction through the occlusive material.

9. The method of claim 8, wherein the surgical excision comprises:
advancing the catheter to a target area in the vascular lumen of the patients in the first and second patient populations, moving the rotating cutter out of the side facing cutting window in the catheter;
deflecting a distal portion of the catheter to urge the cutter toward the occlusive material; and
advancing the cutter through the occlusive material by moving the catheter.

10. The method of claim 8, wherein greater than 50 mg of vascular lumenectomy material is removed for testing from each patient.

11. A method of screening for drug efficacy in a patient having cardiovascular disease comprising:
removing a first sample of lumenectomy material from a first location in a vascular lumen of the patient;
testing the first sample in a first test for the presence or absence or amount of a marker, administering a drug to the patient;
removing a second sample of lumenectomy material from a second location in a vascular lumen of the patient;
testing the second sample in a second test for the presence or absence or amount of the marker; and
evaluating the efficacy of the drug on the basis of comparing the presence or absence or amount of the marker in the first and second tests,
wherein the lumenectomy material comprises an atherosclerotic tissue, a restenotic tissue, or other tissue excised from a vascular lumen.

12. The method as in claim 11, wherein the drug comprises a drug to reverse, prevent, or stabilize a cardiovascular condition.

13. The method of claim 12, wherein the cardiovascular condition comprises atherosclerosis or restenosis.

14. The method of claim 11, wherein the marker comprises an amino acid.

15. The method of claim 11, wherein the marker comprises a nucleic acid.

16. The method of claim 11, wherein the marker comprises a cell surface molecule.

17. The method of claim 11, wherein the marker comprises a marker found in vascular lumenectomy material.

18. The method of claim 11, wherein removing the lumenectomy material comprises percutaneous surgical excision comprising:
providing a catheter having a rotating cutter, a collection chamber, and a cutting window, the collection chamber begin distal to the cutting window, the rotating cutter being movable between a stored position and an exposed position, at least part of the rotating cutter becoming exposed through the cutting window when moving to the exposed position;
exposing the cutter by moving the cutter to the exposed position; and
advancing the catheter in a distal direction to move the rotating cutter through occlusive material in the body lumen, the rotating cutter remaining in the exposed position so that the cutter and the window maintain their orientation with respect to one another when advancing the catheter through the occlusive material, the occlusive material cut by the rotating cutter being directed through the cutting window and distally into the collection chamber as the catheter is advanced in the distal direction through the occlusive material.

19. The method of claim 18, wherein the surgical excision comprises:
advancing the catheter to a target area in the vascular lumen of the patients in the first and second patient populations, moving the rotating cutter out of the side facing cutting window in the catheter;
deflecting a distal portion of the catheter to urge the cutter toward the target occlusive material; and
advancing the cutter through the occlusive material by moving the catheter.

20. A method of claim 11, wherein the presence or absence or amount of more than one marker is tested in the first and second samples.

21. A method of screening for drug efficacy in a population of patients having cardiovascular disease comprising:
removing a first sample of lumenectomy material from a first location in a vascular lumen of at least a first patient and a second patient;
testing the first samples in a first test for the presence or absence or amount of a marker;
administering a drug to the first patient and a placebo to the second patient;
removing a second sample of lumenectomy material from a second location in a vascular lumen of at least the first patient and the second patient;
testing the second samples in a second test for the presence or absence or amount of the marker; and
evaluating the efficacy of the drug on the basis of comparing the presence or absence or amount of the marker in the first and second tests for the first patient administered the drug compared to the second patient administered the placebo,
wherein the lumenectomy material comprises an atherosclerotic tissue, a restenotic tissue, or other tissue excised from a vascular lumen.

22. The method as in claim 21, wherein the drug comprises a drug to reverse, prevent, or stabilize a cardiovascular condition.

23. The method of claim 22, wherein the cardiovascular condition comprises atherosclerosis or restenosis.

24. The method of claim 21, wherein the marker comprises an amino acid.

25. The method of claim 21, wherein the marker comprises a nucleic acid.

26. The method of claim 21, wherein the marker comprises a cell surface molecule.

27. The method of claim 21, wherein the marker comprises a marker found in vascular lumenectomy material.

28. The method of claim 21, wherein removing the lumenectomy material comprises percutaneous surgical excision comprising:
providing a catheter having a rotating cutter, a collection chamber, and a cutting window, the collection chamber begin being distal to the cutting window, the rotating cutter being movable between a stored position and an exposed position, at least part of the rotating cutter becoming exposed through the cutting window when moving to the exposed position;
exposing the cutter by moving the cutter to the exposed position; and
advancing the catheter in a distal direction to move the rotating cutter through occlusive material in the body lumen, the rotating cutter remaining in the exposed position so that the cutter and the window maintain their orientation with respect to one another when advancing the catheter through the occlusive material, the occlusive material cut by the rotating cutter being directed through the cutting window and distally into the collection chamber as the catheter is advanced in the distal direction through the occlusive material.

29. The method of claim 28, wherein the surgical excision comprises:
advancing the catheter to a target area in the vascular lumen of the patients in the first and second patient populations, moving the rotating cutter out of the side facing cutting window in the catheter;
deflecting a distal portion of the catheter to urge the cutter toward the occlusive material; and
advancing the cutter through the occlusive material by moving the catheter.

30. A method as in claim 21, wherein the presence or absence or amount of more than one marker is tested in the first and second samples.

31. The method of claim 18, wherein greater than 50 mg of vascular lumenectomy material is removed for testing from each patient.

32. The method of claim 28, wherein greater than 50 mg of vascular lumenectomy material is removed for testing from each patient.
